(19) United States
(12) Patent Application Publication
(10) Pub. No.: US 2021/0022661 A1
(43) Pub. Date: Jan. 28, 2021

(54) INTRAVAGINAL SYSTEM FOR MENSTRUAL CYCLE MONITORING

(71) Applicant: PreOV, LLC., Salt Lake City, UT (US)

(72) Inventors: Jeanna Ryan, Salt Lake City, UT (US); Joni Aoki, Salt Lake City, UT (US); Young Hong, Salt Lake City, UT (US); Ronald Heffernan, Sandy, UT (US); June Chen, Sandy, UT (US)

(21) Appl. No.: 17/046,190

(22) PCT Filed: Apr. 9, 2019
(86) PCT No.: PCT/US2019/026573

§ 371 Date: Oct. 8, 2020

Related U.S. Application Data
(60) Provisional application No. 62/655,044, filed on Apr. 9, 2018.

(51) Int. Cl. A61B 5/00; A61B 5/053

(52) U.S. Cl. CPC A61B 5/4337; A61B 5/0008; A61B 5/0022; A61B 5/0537; A61B 5/0538

(57) ABSTRACT

A menstrual cycle monitoring system can be used to monitor fertility. The menstrual cycle monitoring system can comprise an intravaginal body which includes a hydration sensing probe(s) and/or sensor(s) and a temperature sensor oriented to measure bioelectrical impedance, resistance and/or conductance within cervical mucus and/or the vaginal environment and basal body temperature, respectively. A data communication unit stores and optionally transmits collected fertility data which includes at least the measured bioelectrical impedance, resistance and/or conductance and temperature over time. A power source can be electrically coupled to the hydration sensing probe(s) and/or sensor(s), temperature sensor and the data communication unit.

### CROSS-REFERENCE TO RELATED APPLICATION

This application claims priority to and the benefit of the filing date of U.S. Provisional Patent Application No. 62/655,044, filed Apr. 9, 2018, which is incorporated by reference herein in its entirety.

### FIELD

The present invention relates generally to using a probe or sensor device to sense or measure the changes in the parameters of the intravaginal and cervical environment, of the vaginal fluid or cervical mucus quality by impedance, and the temperature of the vagina or cervical vault that can be read wirelessly via a reader device.

### BACKGROUND

The Center for Disease Control reports that one out of eight couples, or 12% of women between the ages 15-44 in the United States (US) have difficulty conceiving. Worldwide, there is expected to be an overall increase in difficulty to successfully conceive significantly widening the market for fertility products. Fertility of the U.S. population is below replacement for those native born. There is also a declining birth rate in Britain, Western European countries and Japan. Initially, decreased birth rates were beneficial to the economy. However, more recently, demographers and economists among developed countries are becoming more concerned with the low birth rates as population replacement has fallen below stability limits, which increases the danger of not being able to replace the aging workforce resulting in economic instability due to effects such as low tax revenue. Japan and France have already implemented pro-family policies to increase birth rates.

Many women track their menstrual cycle to help determine their most fertile period. However, less than 13% of women trying to conceive can accurately identify the day they ovulate (Zinaman et al. (2012), Curr Med Res Opin. 28 (5): 749-54). Tracking this information aids in conception planning through prediction of pending ovulation and may also be used to predict onset of menstruation to either achieve or avoid pregnancy. Current methods to track fertile periods can be heavily subjective, expensive, inconvenient, stressful, and time consuming.

Analysis of cervical mucus is a recommended and effective method for tracking menstrual cycles. Scientific research has demonstrated that estrogen levels and the hydration of cervical mucus peak simultaneously, indicating pending ovulation as illustrated in FIG. 1. In addition, cervical mucus hydration increases several days prior to ovulation, and a woman's hydration pattern remains consistent among menstrual cycles. Cervical mucus >97.5% water provides an environment conducive for sperm to survive in order to fertilize the egg. A small amount of sperm can live for up to 5 days, but the optimal timing of intercourse is around 2 days prior to ovulation. Therefore, predicting ovulation several days in advance gives women and couples time to plan ahead for their most fertile period. Products currently available on the market that are used for ovulation prediction such as urine luteinizing hormone tests and basal body temperature monitoring provide women with less than 24 hours' notice prior to ovulation (Su et al. (2017), Bioeng Transl Med. 2 (3): 238-246).

A large number of studies have been conducted demonstrating the efficacy of monitoring cervical mucus for identifying the fertile period in order to increase the probability of conception (Bigelow et al. (2004), Human Reproduction. 19 (4): 889-892). The success rate for these methods if used correctly has been found to be 97% with typical use rate of failure being 15.9% (Mayo Clinic, 2019). Furthermore, monitoring basal body temperature can be used for ovulation detection. When cervical mucus and basal body temperature monitoring are used simultaneously, pregnancy is prevented at a rate of 99.6% with correct use (Frank-Hermann et al. (2007), Human Reproduction. 22 (5): 1301-1319). The typical user failure rate may be partly due to the difficulty in obtaining and analyzing cervical mucus. Current methods of cervical mucus collection for ovulation detection require manual extraction and rely on individuals to visually analyze the mucus viscosity and texture. This method is highly subjective, may not be performed at the right time, and may be uncomfortable or unacceptable for many women. Additionally, a woman's basal body temperature frequently fluctuates and variable methods to obtain measurements of temperature can often lead to inaccurate results and analysis. Regardless, monitoring the menstrual cycle continues to be a significant challenge in terms of reliability and convenience.

Women tracking their menstrual cycle seek products that are accurate and clearly communicate results. In addition, busier lifestyles have driven the need for devices that are convenient, discrete, time saving, and easy-to-use. The device allows the monitoring of cervical mucus without the user having to extract cervical mucus manually, collect a urine sample, or measure their body temperature repeatedly. With more technology capabilities available, women also seek products that can be synchronized with their smartphones and computers.

### SUMMARY

A real-time menstrual cycle monitoring system can be used to monitor fertility. The menstrual cycle monitoring system can comprise an intravaginal body which includes both a hydration sensing probe(s) or sensor(s) oriented to measure bioelectrical impedance, resistance or conductance of cervical mucus or the vaginal and cervical environment, and a temperature sensor to monitor body temperature. A data communication unit stores and transmits collected fertility data. A power source can be electrically coupled to the hydration sensing probe(s) or sensor(s) and the data communication module.

The intravaginal sensor device may have a body in the shape of a ring, a torus, or a cylinder that may or may not be elongated. In one example, the intravaginal body can be an annular ring body sized to be oriented within the vagina, cervical environment or vaginal vault. In one example, the annular body is formed of polyurethane. Although the annular body can often be a single seamless body, in one example, the annular body includes a seam configured to provide access to the power source. Sections of the body may or may not be hollow to allow housing of any or all other components, including electrical.

The hydration sensing probe(s) or sensor(s) can include a complementary set of electrodes oriented in proximity to one another sufficient to measure bioelectrical impedance, resistance or conductance associated with cervical mucus, vaginal or cervical tissue or epithelium, or the cervical environment. In one example, the complementary set of electrodes have a gold electrode surface. In yet another alternative, the hydration sensing probe(s) or sensor(s) further includes an antimicrobial coating.

The data communication unit further comprises a wireless transmitter configured to send the fertility data to a mobile device, computer or other platform. In this manner, the collected data can be immediately communicated to a user through a wireless data transfer. A mobile receiving device (e.g. a smartphone) can then use an associated tracking application which analyzes and displays fertility estimates based on the collected fertility data.

The temperature sensor can be configured to measure temperature and communicate the measured temperature to the data communication unit via radio frequency fields or Bluetooth. The temperature sensor may have measuring accuracy of ±0.1° C. However, these measurement ranges may be varied as desired for the application of the invention. Such data can supplement the calculation of fertility window estimates.

There has thus been outlined, rather broadly, the more important features of the invention so that the detailed description thereof that follows may be better understood, and so that the present contribution to the art may be better appreciated. Other features of the present invention can become clearer from the following detailed description of the invention, taken with the accompanying drawings and claims, or may be learned by the practice of the invention.

### BRIEF DESCRIPTION OF DRAWINGS

FIG. 1 is a graph showing timing of Ovulation in Relation to % Hydration of Cervical Mucus, Luteinizing Hormone (LH), and Basal Body Temperature (BBT).

FIG. 2 is a bar graph illustrating fertility statistics.

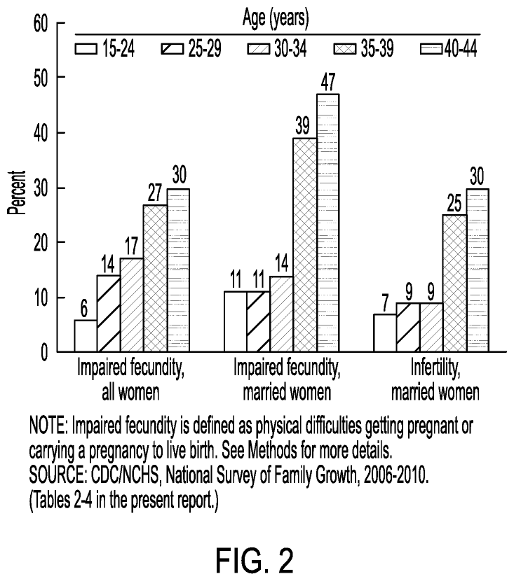

FIG. 3 is a cervical mucus and fertility correlation algorithm according to one aspect of the disclosure.

FIG. 4A is a perspective view of an exemplary menstrual cycle monitoring device as disclosed herein.

FIG. 4B is a schematic diagram depicting components of an exemplary menstrual cycle monitoring system as disclosed herein.

FIG. 4C is a schematic diagram depicting an exemplary environment for a remote computing device as disclosed herein.

FIG. 5 is a graph illustrating the inverse relationship of sodium content and measured electrical resistance of cervical mucus.

FIG. 6 is a diagram illustrating components of an operating environment in which various aspects of the disclosure may be implemented.

These drawings are provided to illustrate various aspects of the disclosure and are not intended to be limiting of the scope in terms of dimensions, materials, configurations, arrangements or proportions unless otherwise limited by the claims.

### DETAILED DESCRIPTION OF THE INVENTION

While these exemplary embodiments are described in sufficient detail to enable those skilled in the art to practice the invention, it should be understood that other embodiments may be realized and that various changes to the invention may be made without departing from the spirit and scope of the present invention. Thus, the following more detailed description of the embodiments of the present invention is not intended to limit the scope of the invention, as claimed, but is presented for purposes of illustration only and not limitation to describe the features and characteristics of the present invention, to set forth the best mode of operation of the invention, and to sufficiently enable one skilled in the art to practice the invention. Accordingly, the scope of the present invention is to be defined solely by the appended claims.

### Definitions

In describing and claiming the present invention, the following terminology can be used.

The singular forms “a,” “an,” and “the” include plural referents unless the context clearly dictates otherwise. Thus, for example, reference to “an electrode” includes reference to one or more of such electrodes and reference to “a subject” refers to one or more such subjects.

As used herein, the term “about” is used to provide flexibility and imprecision associated with a given term, metric or value. The degree of flexibility for a particular variable can be readily determined by one skilled in the art. However, unless otherwise enunciated, the term “about” generally connotes flexibility of less than 5%, and most often less than 1%, and in some cases less than 0.01%.

As used herein with respect to an identified property or circumstance, “substantially” refers to a degree of deviation that is sufficiently small so as to not measurably detract from the identified property or circumstance. The exact degree of deviation allowable may in some cases depend on the specific context.

As used herein, “adjacent” refers to the proximity of two structures or elements. Particularly, elements that are identified as being “adjacent” may be either abutting or connected. Such elements may also be near or close to each other without necessarily contacting each other. The exact degree of proximity may in some cases depend on the specific context.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

As used herein, the term “at least one of” is intended to be synonymous with “one or more of.” For example, “at least one of A, B and C” explicitly includes only A, only B, only C, and combinations of each.

Concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a numerical range of about 1 to about 4.5 should be interpreted to include not only the explicitly recited limits of 1 to about 4.5, but also to include individual numerals such as 2, 3, 4, and sub-ranges such as 1 to 3, 2 to 4, etc. The same principle applies to ranges reciting only one numerical value, such as “less than about 4.5,” which should be interpreted to include all of the above-recited values and ranges. Further, such an interpretation should apply regardless of the breadth of the range or the characteristic being described.

Any steps recited in any method or process claims may be executed in any order and are not limited to the order presented in the claims. Means-plus-function or step-plus-function limitations will only be employed where for a specific claim limitation all of the following conditions are present in that limitation: a) “means for” or “step for” is expressly recited; and b) a corresponding function is expressly recited. The structure, material or acts that support the means-plus function are expressly recited in the description herein. Accordingly, the scope of the invention should be determined solely by the appended claims and their legal equivalents, rather than by the descriptions and examples given herein.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

Menstrual Cycle Monitoring Device and System

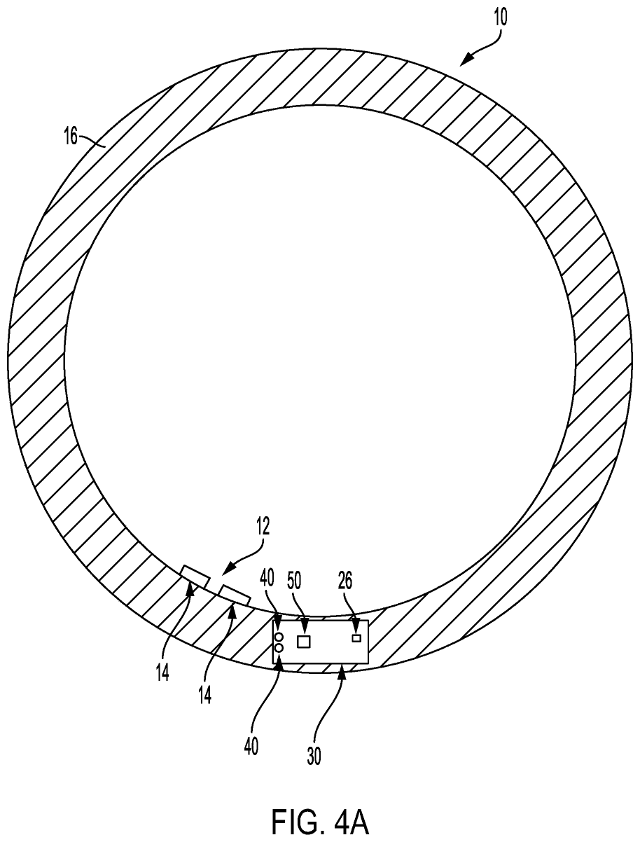

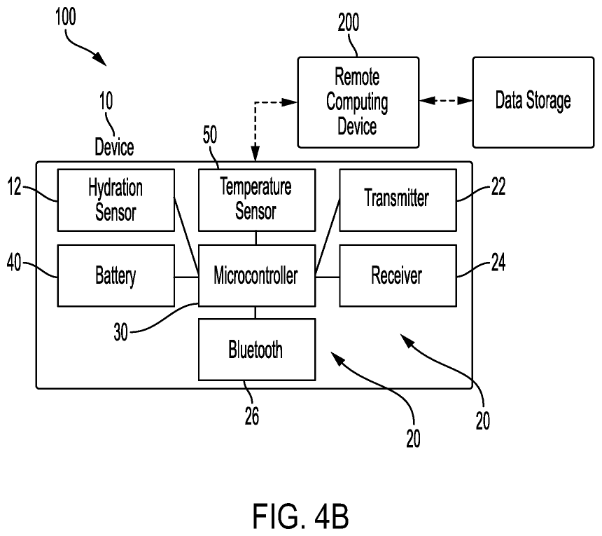

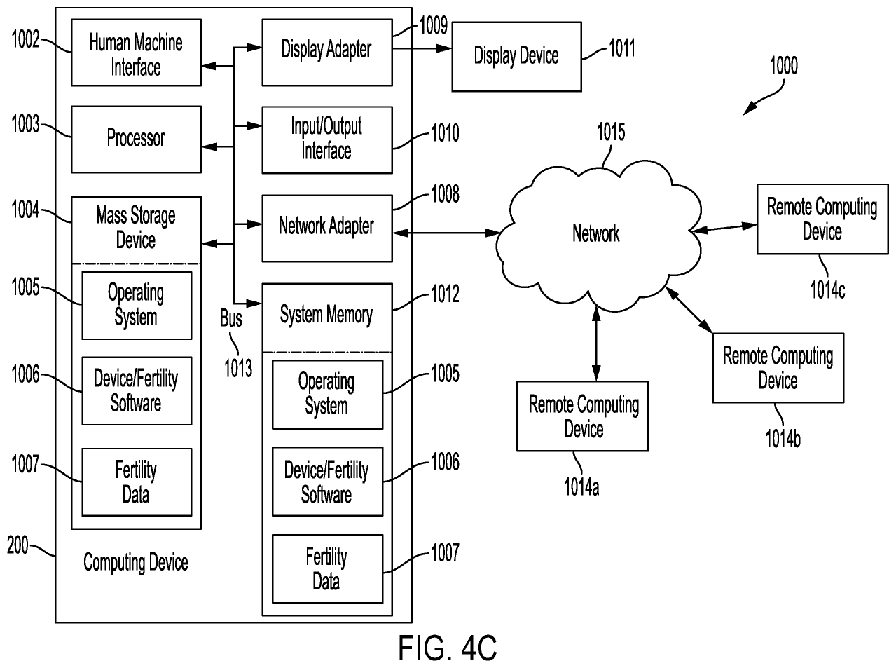

With reference to FIGS. 4A-4C, the disclosed fertility tracking and menstrual cycle monitoring system 100 can overcome one or more of these barriers by providing reliable and real-time information in an easy-to-use and convenient approach. The system 100 is intended to be used by women trying to conceive and/or to prevent pregnancy and/or to monitor the menstrual cycle. The system 100 can help women predict their peak fertile period based on the hydration and/or ion levels of their cervical mucus and cervical environment. The product can be inserted into the vagina up to the cervix (but not into the cervix) following the last day of menstruation, and removed once ovulation has occurred. The number of days used may vary depending on the length of the user's menstrual cycle, which can vary. Average length of use per month is around 7 to 14 days, however, it can be worn for up to 30 days or maybe longer.

Throughout the menstrual cycle, various hormones fluctuate in order for ovulation or release of an egg to occur. Hydration of cervical mucus changes depending on blood levels of estrogen and progesterone. As blood levels of estrogen increase, cervical mucus hydration increases. Surge in estrogen causes the LH surge stimulating ovulation.

The system 100 and associated processing circuitry (which can execute a software application as further disclosed herein), can monitor and track hydration and/or ion levels of cervical mucus and the vaginal environment to predict the estrogen surge prior to the LH surge and pending ovulation. Hydration of cervical mucus can begin to increase greater than 2% from baseline 3-5 days prior to the peak in LH. A % hydration of cervical mucus greater than 97.5% can indicate ideal sperm penetrability. Two days prior to peak LH, % hydration of cervical mucus is expected to be around 98%. The disclosed processing circuitry, through the software application and a remote display as further disclosed herein, can display fertile days for timing of intercourse with a range of possible fertility to high fertility using one or more of a plurality of indicators, including but not limited to color, symbols, or any other method(s). Menstruation would be expected to start at around 14 days after ovulation and, depending on the individual's cycle length, can also be indicated to the user by the disclosed processing circuitry, which can be configured to execute the software application.

As shown in FIGS. 4A-4B, an intravaginal ring 10 can be integrated with a hydration and/or ion sensor technology 12. The hydration and/or ion sensor(s) 12 may be composed of probe(s) and/or electrodes measuring the electrical impedance, resistivity and/or conductance of cervical mucus, the vaginal environment, and/or fluids and secretions near the cervix. The probe(s) and/or sensor(s) 12 can be at least partially embedded in the ring 10 and can optionally comprise a pair of electrodes 14, which can optionally be circumferentially spaced about the ring 10. In exemplary configurations, the electrodes 14 can project from a surface of the ring body 16 as shown in FIG. 4A. A voltage can be applied between electrodes 14, and the drop in voltage corresponds to a measurement of the resistance of fluids, which may be converted to impedance. Electrical impedance is an extension of the concept of resistance and measures the opposition that a circuit presents to a current when a voltage is applied. Impedance possesses both magnitude and phase. In one specific example, a sinusoidal strength current of 5 kHz can be passed through the cross-sectional area, which can determine extracellular fluid content based on the signal frequency and calculate and evaluate variations in hydration and/or ion levels. At low frequencies such as 5 kHz, current flows primarily through the extracellular water (ECW). An alternating current can be applied to measure electrical resistance and conductance of cervical mucus as they are related to the content of water and characteristic electrolytes, particularly sodium. As ovulation approaches, both the volume of water and sodium content in cervical mucus increase and the concurrent decrease in resistance can be objectively measured.

A hydration and/or ion sensor 12 can be in the form of a capacitor which includes two interdigitated or parallel electrodes 14 that are made of a suitable conductor, e.g. gold. The hydration and/or ion sensor 12 can be operated at a suitable frequency such as 5 kHz. However, it is contemplated that any desired optimal sensor frequency can be used. For example, it is contemplated that a first frequency can be optimal for applications where measurements of cervical fluid are performed, and a second frequency can be optimal for other applications where measurements of tissue (e.g., vaginal and/or cervical epithelium/tissue) are performed. Probes and/or electrodes may be covered or made with an antimicrobial or hygienic promoting coating such as pMTAC or pDA-g-pMTAC combo, silver, tin, copper, ZnO/Ti spray, DMDC-Q-g-EM hydrogel, platinum, titanium, alloys, stainless steel, cobalt or cobalt-based alloys, cobalt chromium, magnesium alloys, or other material that is biocompatible. It is contemplated that spectroscopy can also be used for cervical mucus and/or vaginal environment analysis. Estimates of hydration, percent water content, and/or other estimates can be generated from the data collected by the probe(s). Other possible data collections concerning cervical mucus and/or the vaginal environment may include viscosity, pH, osmolarity, MUC 4 protein, MUC 5B protein, copper, iron protein:glycol ratio, IgA, IgG, Lactoferrin, Interleukin-10 and/or other antimicrobial peptides.

As shown in FIGS. 4B-4C, real time data from the sensors 12 can be collected and translated to an external (remote) computing device 200 via a data communication unit 20, which can include Bluetooth, radio-frequency identification (RFID), telemetry or other wired or wireless technology. In these aspects, it is contemplated that the data communication unit 20 can comprise a wireless transmitter 22 and a wireless receiver 24. Optionally, it is contemplated that the wireless transmitter 22 and the wireless receiver 24 can be provided as a single component (e.g., a wireless radio or transceiver). In further exemplary aspects, it is contemplated that the data communication unit 20 can comprise Bluetooth hardware as is known in the art. In still further aspects, the data communication unit 20 can be communicatively coupled to a microcontroller 30, which can, in turn, be communicatively coupled to the sensors 12/electrodes 14 and the temperature sensor 50 as disclosed herein. In these aspects, it is contemplated that the microcontroller can receive fertility data from the sensors 12, 50 and then direct the transmitter to transmit the fertility data to the processor 1003 of the remote computing device 200. The data can be integrated with a software application accessible by the remote computing device 200 (e.g., a computer, smartphone, smart watch, tablet, or other mobile device or other software application device) for data storage, analysis and/or menstrual cycle tracking. The software application, when executed by a processor 1003 of the remote computing device 200, can aid the user in identifying the most fertile days of their menstrual cycle and provide information regarding the individual's menstrual cycle, including, for example and without limitation, an estimated time of ovulation. In operation, it is contemplated that the wireless transmitter 22 of the data communication unit 20 can transmit fertility data to the processor 1003 of the remote computing device 200, while the wireless receiver 24 of the data communication unit 20 can receive remote instructions from the processor 1003 of the remote computing device.

With reference to FIG. 4A, the intravaginal ring 10 can be made of flexible, selectively deformable material, which may include plastic or other material or polymer such as medical-grade polyurethane, silicone, ethyl vinyl acetate, or other biocompatible polymer. The core (i.e., the interior of the ring body 16) may or may not be hollow depending on the internal contents that may include a battery, wiring, or other necessary contents to power the device and allow it to communicate data to the software application (referred to collectively as power source 40). The ring 10 may generally have an outside diameter between 55 mm and 65 mm but may reach up to 120 mm. The ring 10 may generally have an inner diameter of 45 mm but may range between 34 mm to 65 mm. Alternatively, the diameter may be different (vary) along the circumference of the ring. Pliability or flexibility of the ring 10 can generally be between 0.01 to 3.00 Newtons. Tensile strength may be at least 500 psi but may range between 115 to 5400 psi. The ring 10 may have a twist angle of up to 55 degrees and shall return to the original diameter and elasticity. The cross-sectional diameter may have a measurement of 10 mm but may vary between 3 mm to 30 mm. The compression resistance shall be 55% to 85% of its original diameter. The ring 10 may be a single continuous piece or broken at the seam(s) (not shown) and/or at the location(s) of the battery to allow removal of the battery or other components for proper disposal.

The device 10 according to the invention for measuring both electrode impedance and temperature can contain electrodes 14 that are spaced apart by a certain distance which may generally range between 1 mm and 5 mm but may reach up to 65 mm from one another. The device 10 may further comprise a temperature sensor 50 configured to measure body temperature. In use, the electrodes 14 can provide a signal indicative of electrode impedance to a data communication unit 20 as further disclosed herein. Similarly, the temperature sensor 50 can communicate the measured body temperature (or provide a signal indicative of the measured body temperature) to the data communication unit 20. Optionally, the microcontroller 30 can receive data from the electrodes 14 and/or temperature sensor 50 and then cause the data communication unit 20 (e.g., the wireless transmitter 22) to transmit the data to the remote computing device 200.

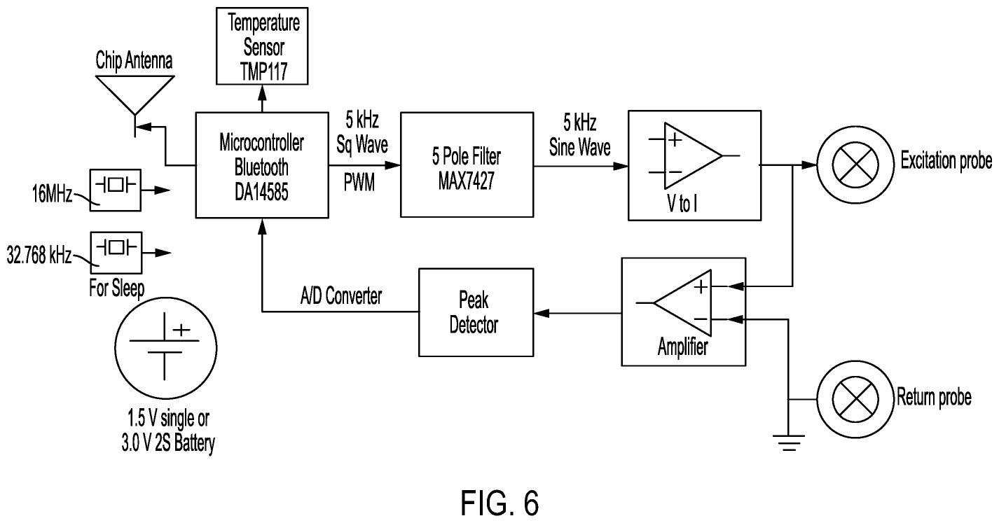

The device can be connected to a current source and to the amplifier. With reference to FIG. 6, it is contemplated that during impedance measurements, the microcontroller 30 can generate a square wave using a pulse width modulation peripheral. This signal can be passed to a capacitor filter integrated circuit to generate a sine wave. The sine wave is fed to a transconductance amplifier which converts the voltage signal to the desired current, which is then applied to the excitation electrode through a suitable AC coupling capacitor (which blocks any DC component of the signal). The resulting voltage can be sensed by a high impedance differential or instrumentation amplifier, and the sensed voltage can be applied to a peak detector circuit which can convert the AC to a DC peak value that is then measured using the Analog-to-Digital Converter peripheral in the microcontroller 30.

The ring 10 can be manually inserted into the vagina close to the cervix at the end of the menstruation period, which is usually around Day 7 of the menstrual cycle for most users. The ring 10 may be worn until the day of ovulation and/or the start of menstruation, which is around Days 14 and 28, respectively, of the menstrual cycle for most users. Cervical mucus changes in response to changes in estrogen, progesterone, and/or other biomarkers and their ratio, which change once implantation has occurred. In one alternative aspect, the ring 10 may be used to measure hydration, change in rheological properties, volume, viscosity, and/or conductance of cervical mucus and/or the vaginal environment, and/or early pregnancy factor in cervical mucus to indicate early pregnancy soon after implantation, which may occur anywhere from seven to twelve days after ovulation.

This ring or device 10 can provide a reliable, convenient and easy-to-use tool for women and couples trying to conceive, prevent pregnancy, or track the menstrual cycle. In use, the sensor-integrated intravaginal ring 10 can provide real time objective measurements of cervical mucus and the vaginal or cervical environment. In order to increase chances of conceiving or preventing pregnancy, this information can be integrated into a software application to help users track their ovulation window (most fertile days) and menstrual cycle.

As previously mentioned, the ring 10 has wireless communication capabilities to present real-time and quantitative measurements of the impedance, resistance or conductance of cervical mucus and/or the vaginal environment. Optionally, such quantitative measurements can be correlated to a percent water content or other easy to understand measurement that is displayed to the user. The impedance, resistance or conductance data gathered from the probe(s) and or sensor(s) 12 can be collected automatically and wirelessly transmitted to an external software application with an algorithm to chart the user's menstrual cycle, e.g. typically in a calendar, graph and/or other format(s). The calendar can be accessed through the user's smartphone, smart watch, tablet, computer and/or other software application device (i.e., a remote computing device 200). Through monitoring of cervical mucus and the vaginal environment, the disclosed system 100 provides more advanced notice of pending ovulation compared to commonly used fertility monitors. Advance notice can be up to 5 days' notice (e.g., up to 48, 72, 96, or 120 hours in advance).

Default manufacturer settings for measuring impedance, resistance or conductance can be at any fixed interval, including every 15 or 30 minutes (resulting in 48-96 readings per day), every 1-2 hours (resulting in 12-24 readings per day), or every 3-6 hours (resulting in 4-8 readings per day). However, the user may or may not adjust these measurement settings to as frequent as every hour to as infrequent as no measurements (e.g. and rely on a manual request to the device, through the wireless receiver and microcontroller, to take a reading). Optionally, the measurement settings can be adjusted to modify the frequency of impedance, resistance or conductance measurements throughout the day and/or to have a variable interval. For example, it is contemplated that impedance, resistance or conductance measurements can be taken at one interval (e.g., every 30 minutes) during certain hours or times of day and taken at a second interval (e.g., every 3 hours during certain hours or times of day). The user may or may not also take a reading at any point they request through the software application interface. Once impedance, resistance or conductance measurements indicate that cervical mucus hydration has exceeded a hydration threshold (e.g., increasing by an absolute value of >2% or reaching >97% hydration), the processor (through the software application) can provide an output to the user (e.g., a visual output on a display) that is indicative of pending ovulation. Optionally, the device 10 can be configured to perform measurements in a continuous, automated fashion at the intervals discussed above. Optionally, the device 10 can permit user-initiated (on-demand) measurements; however, it is understood that the device need not permit such user-initiated measurements.

More specifically, cervical mucus contains water, electrolytes, other ions, and other particles. When a current is passed through a fluid, it is able to conduct electrical current. At low frequencies at around or below 5 kHz, current from the electrodes 14 primarily passes through the extracellular fluid space (Weyer et al. (2012), Acta Polytecha. 52 (5): 120-124), but begins to penetrate body tissues as frequency increases. A fixed or variable sinusoidal strength current can be passed through the cross-sectional area of the tissue in proximity to the device 10. As current is passed through the tissue, fluid and ion resistance or conductance can be determined based on the signal frequency as further disclosed herein, and this resistance or conductance can then be correlated with a corresponding variation in hydration levels as further disclosed herein.

Monitoring cervical mucus is a highly effective method, but unlike conventional methods of monitoring cervical mucus, the disclosed device 10 is intended to remain in the vagina for an extended period of time (e.g., 14 days) to specifically measure and/or determine the hydration of cervical mucus as one of its fertility biomarkers. The sharp increase in hydration of cervical mucus indicates pending ovulation, and it also provides an optimal environment for sperm penetrability. Additionally, the disclosed system provides women and couples with more advanced notice of pending ovulation, which is crucial for planning around the brief fertile window.

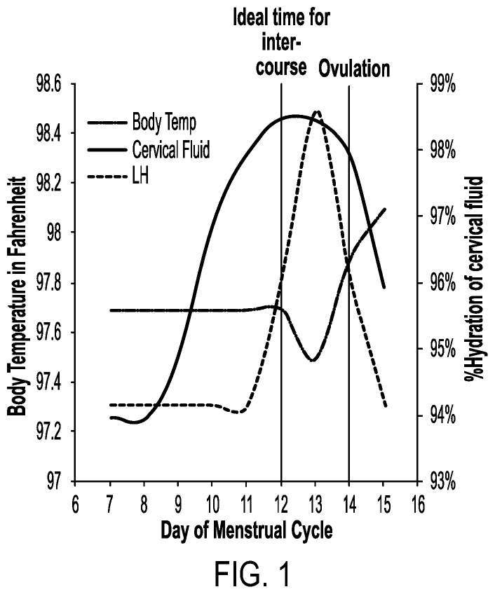

Users do not need to manually upload or interpret data, as the wireless capabilities of the device allow for automatic communication with cell phones, mobile devices (e.g., tablets, smart watches) and/or other computer applications but may manually input information. The disclosed system provides personalized menstrual cycle information giving women and couples more time to plan and time intercourse as the disclosed system predicts ovulation up to 5 days prior. In contrast, conventional monitoring of other biomarkers like peak LH in the urine or basal body temperature identifies ovulation less than 24 hours in advance as can be seen in FIG. 1.

Normal internal body temperature is maintained between 36.5-37.5° C. (97.7-99.5° F.), while relative humidity inside the body is 100%. Silver oxide, zinc alkaline, and other batteries having good resistance to shock and vibration can be adequate for the disclosed device and system.

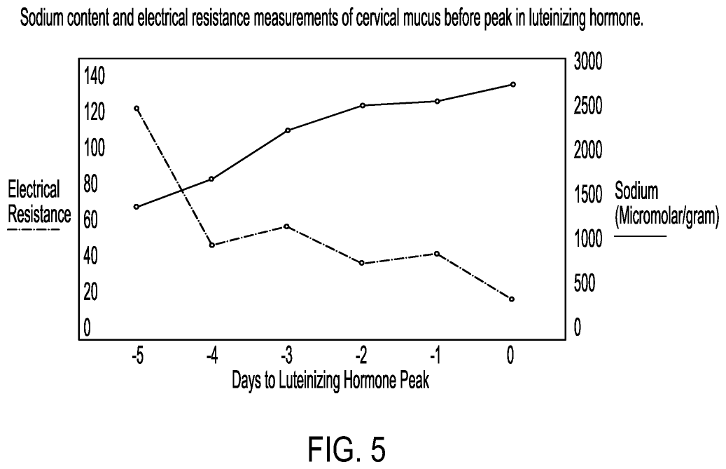

Furthermore, the cervix is a firm, cylindrical structure situated at the lower pole of the uterine corpus. The non-pregnant cervix is about 25 mm in length, with an anteroposterior diameter ranging between 20-25 mm and a transverse diameter of 25-30 mm. Considerable variations may exist depending on age, parity and stage of menstrual cycle. Notably, the main product of the human cervix is cervical mucus, which is manufactured and secreted by the columnar cells in the cervix throughout the menstrual cycle and is regulated by estrogen and progesterone. The cervical mucus that is produced during ovulation has a stretchy and stringy consistency. Following ovulation, mucus reduces in fluidity and volume and becomes viscous. These changes can be correlated with changes in hydration and ion levels. Cervical mucus displays characteristic changes in water and electrolyte content throughout the menstrual cycle. As shown in FIG. 5, sodium content increases and measured electrical resistance of cervical mucus decreases during the 5 days leading up to ovulation (Day 0), which is when luteinizing hormone (LH) also reaches maximal level. As sodium concentration and/or content increases, the electrical resistance decreases. Thus, it is contemplated that the water content and/or viscosity changes of cervical mucus and/or hydration changes to the cervical and/or vaginal epithelium that occur leading up to ovulation also contribute to this electrical resistance pattern. Accordingly, as the resistance of cervical mucus changes, it is understood that a corresponding change to the hydration of the cervical mucus also occurs. In exemplary aspects, it is contemplated that the disclosed processor, through a software application, can monitor recorded fertility data to determine when measured resistance, impedance, or conductance exceeds a threshold value that corresponds to a change in cervical mucus hydration that is indicative of fertility.

The ring 10 can communicate with the user's accessory interface using Bluetooth 4.0, also known as Bluetooth Low Energy (BLE). For example, IEEE Std 802.15.4-2015 is one current suitable protocol while compatible interconnection for data communication devices using low-data-rate, low-power, and low-complexity short-range radio frequency (RF) transmissions in a wireless personal area network (WPAN).

Although numerous materials can be used for the device body 16, the inherent nonconductive characteristic of polyurethane, and its physical-mechanical properties, surpasses the performance of latex and other materials. Medical-grade polyurethanes fall under the “prolonged” exposure category, also called “healthcare grade,” based on tests such as hemolysis, genotoxicity, and intramuscular implantation with histopathology. Medical-grade polyurethane exhibits great tensile strength, excellent elongation, superior flexibility, versatility, low compression set and a durometer range (hardness) of 10 to 100 Shore A. The material has a surface or implant contact of more than 24 hours and 30 days. Polyurethanes have demonstrated long-term biocompatibility in many biomedical and drug delivery applications and are approved for medical applications for conditions under which it can come into contact with skin due to its tremendous stability and water-repellent properties. This prohibits cured material from being affected by contact with skin. It is also resistant to bacterial and fungal growth. The battery 40, Bluetooth hardware 26, and other components can be fully enclosed within the intravaginal ring core and completely covered by medical grade polyurethane preventing exposure to bodily tissue or fluids. Gold metal used for the hydration sensors is also widely used in biomedical devices due to its biocompatibility, inertness within the body, and bio-robustness.

Under normal conditions of use, the battery 40 is hermetically sealed within the core of the ring body 16. The battery 40 and Bluetooth components 26 can typically be fully enclosed within the intravaginal ring core and completely covered by medical grade polyurethane preventing exposure to bodily tissue or fluids. At low frequencies below approximately 10 kHz, current will not pass across the cell membrane due to the capacitive nature of the bilipid membrane structure.

In use, it is contemplated that the device 10 can be worn during or removed prior to intercourse. It is further contemplated that the device 10 can be removed during menstruation and comfortably worn as long as the ring has been inserted far enough and as close to the cervix as possible. Data from the cervical ring can be communicated to a user-friendly software application accessible by remote computing device 200 or website for analysis and interpretation.

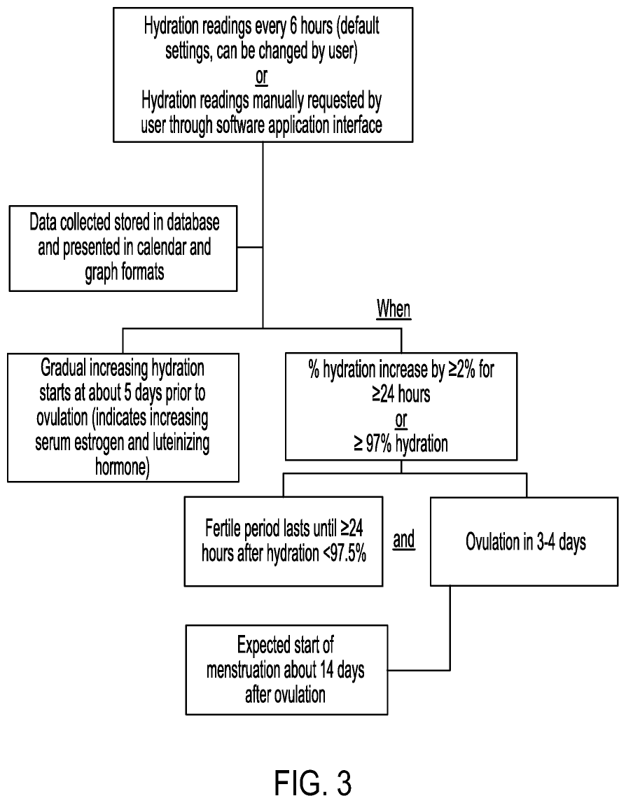

The device can be optimized for fertility and menstrual cycle monitoring. A menstrual cycle algorithm programmed into a software application can analyze the data from the vaginal ring and deliver easily understandable information through a user-friendly interface to help the user identify their most fertile days. FIG. 3 illustrates one example algorithm. Hydration of cervical mucus can be monitored once every 6 hours when default settings are used, but could be measured more often or less often depending on how the firmware is programmed. Information on the user's menstrual cycle can be presented to the user on a display in a format such as a calendar or graph. Hydration of cervical mucus begins to gradually increase at about 5 days prior to ovulation and indicates the concurrent increase in serum estrogen. Optionally, in exemplary aspects, fertile cervical mucus can be identified either when percent hydration increases at a rate greater than 2% within a 24-hour period or when hydration is greater than 97%. The fertile period ends when hydration decreases by greater than 2%, which indicates ovulation has occurred. Optionally, In exemplary aspects, it is contemplated that a decrease in resistance and/or impedance of greater than 5% during a 24-hour period (or other threshold decrease (i.e., percentage decrease or decrease below a specific resistance or impedance value)) can be indicative of or correspond to a greater than 2% increase in percent hydration, thereby causing the processor, through the software application, to provide an output to a user (optionally, through the display device) indicative of a period of fertility. See Fernando et al. (1987), Fertility and Sterility. 47 (3): 409-415. It is further contemplated that an increase in conductance and/or ion (e.g., sodium) levels above a selected threshold increase (i.e., percentage increase or increase below a specific conductance or ion level) can be indicative of or correspond to a greater than 2% increase in percent hydration, thereby causing the processor, through the software application, to provide an output to a user (optionally, through the display device) indicative of a period of fertility. Although specific threshold values are disclosed herein, it is understood that the precise correlation between the decrease in resistance and/or impedance and the increase in percent hydration can vary significantly depending upon the specific patient. Thus, exemplary thresholds that can trigger the signaling of a fertile period can include, for example and without limitation, a decrease in resistance and/or impedance of at least 1%, at least 2%, at least 3%, at least 4%, at least 5%, at least 6%, at least 7%, at least 8%, at least 9%, or at least 10% during a 24-hour period (in comparison to the measured or baseline resistance and/or impedance at the beginning of the 24-hour period). Similarly, it is understood that the precise correlation between the increase in conductance and/or sodium (or other ion) levels and the increase in percent hydration can vary significantly depending upon the specific patient. Thus, other exemplary thresholds that can trigger the signaling of a fertile period can include, for example and without limitation, an increase in conductance and/or sodium (or other ion) levels of at least 1%, at least 2%, at least 3%, at least 4%, at least 5%, at least 6%, at least 7%, at least 8%, at least 9%, or at least 10% during a 24-hour period (in comparison to the measured or baseline resistance and/or impedance at the beginning of the 24-hour period). The expected start of the menstrual cycle is about 14 days after ovulation in a 28-day menstrual cycle (period). The specific algorithms disclosed herein are merely exemplary, and it is specifically contemplated that other algorithms may be used. No concurrent use of other intrauterine, vaginal, or cervical devices is needed or recommended when using this device.

As a general guideline, the device can be a once-a-month use, disposable, and highly portable fertility device. The device, in one example, can consist of an electrified annular cervical ring fabricated of medical-grade, flexible, soft, opaque polyurethane measuring 55 mm in outer diameter, 45 mm in inner diameter, and 10 mm thickness. However, the internal diameter of the ring can match that of the cervix and can have an external diameter that is large enough to wedge the ring against the pelvic floor.

Hydration can most often be monitored once every 15 or 30 minutes, every 1-2 hours, or every 3-6 hours when default settings are used, but can be measured more often or less often as further disclosed herein. Measurements may or may not be requested by the user through the software application as desired. The device can also detect/indicate real-time cervical mucus hydration changes from baseline.

At least one miniaturized electric cell of a plurality of such cells can be interconnected to a miniature battery 40 which is enclosed within the body of the annular ring member. For example, button cell batteries can be suitable such as, but not limited to, SR626 W or equivalent Energizer 377/376. In one specific example, a zinc cathode (negative electrode), a silver oxide anode (positive electrode), and an alkaline electrolyte can be used. Typically, such cells can provide amperage: 28 mAh (to 1.2 volts), impedance (40 Hz): 15-30 ohms, battery duration 35 days or 840 hours, and silver oxide cells have 90% service maintenance after 1-year storage at 21° C. A boost convertor may be used with single 1.5 V batteries; this may be adequate to power the measurement electronics as well. If not, then a separate boost converter may be incorporated.

Optionally, the hydration sensor 12 can be a pair of probes (e.g., electrode probes) installed on one end, two ends, or at any other points throughout the ring 10 with an interdigital spacing of up to 6 mm. Bioelectrical impedance at frequencies of 1-5 kHz has been used to estimate extracellular fluid volume. This device 10 can conduct a frequency of 5 kHz to estimate extracellular fluid volume and analyze this data in an algorithm to evaluate variations in hydration throughout the cycle period. Bioelectric Impedance Analysis (BIA) is the most used and is one of the earliest proposed methods for the estimation of body compartments. BIA, which is based on the inverse proportion between assessed impedance and total body water (TBW), represents the conductive path of the electric current. BIA can estimate water content by using the electrical properties of living tissue. Tissue impedance is proportional to the fluid content when an alternating electrical current is applied and cells acts as capacitors due to the polarity of cell membranes. Cell membrane impedance depends on the frequency of the applied current. At low frequencies, conductance is governed primarily by extracellular water (ECW) as current does not flow through a capacitor but instead flows freely through the ECW. Additionally, conductance and measured impedance of fluids are significantly affected by the concentration of ionic species within a cell. The cyclic variation in the resistance and impedance of cervical mucus can be attributed to the characteristic increase and decrease in the concentration of water and electrolytes such as sodium and chloride in cervical mucus that occur during the menstrual cycle. Specifically, water content of cervical mucus is maximal and thus resistance is minimal at ovulation. The method of measuring the approaching ovulation can be obtained by monitoring changes in cervical mucus secretion and of its impedance by the electrodes as described above. Thus, as measured resistance decreases at or below a threshold value or at or above a threshold rate, the processing circuitry disclosed herein can be configured to provide an output indicative of approaching ovulation.

Body Area Sensor Networks (BASNs) can include signals collected by sensors which relay them to the sink node and are connected to a central interface device. The communications between sensor nodes usually employ wireless technologies like Bluetooth over IEEE 802.15.4. 28 which operates in the 2.4 GHz radio band. BIA is considered safe. Currents at a frequency of 50 kHz are reported to be unlikely to stimulate electrically excitable tissues, such as nerves or cardiac muscle, and relatively small current magnitudes are involved (<1 mA), less than the threshold of perception. Furthermore, the use of batteries or low-voltage power sources greatly diminishes risks from macroshock.

In order to begin use of the device, the user will need to successfully complete a series of steps. After removing the device from product packaging, the user will need to activate the battery (or other power source) 40 prior to use. Such activation can occur using conventional magnetic activation processes or by the removal of pull tabs to permit formation of a closed circuit in the manner known in the art. The user can then hold the device between a thumb and index finger and gently push the sides of the device together to selectively shape the device for insertion into the vagina. The device can then be inserted into the vagina and gently pushed upwards with the index finger until the device reaches the cervix. In use, as further disclosed herein, the device can measure cervical mucus impedance, resistance, or conductance until the device is removed from the cervical environment. Once the user is ready to remove the device, the device can be removed by inserting the thumb and index finger into the vagina and pulling out the device.

FIG. 4C shows an exemplary computing system 1000 that can be used to receive fertility data and/or control operation of various aspects of the disclosed system 100, including the timing of fertility data measurements and the processing of the fertility data. Computing system 1000 can include remote computing device 200 and a display 1011 in electronic communication with the remote computing device, which can be any conventional computing device, such as, for example and without limitation, a personal computer, computing station (e.g., workstation), portable computer (e.g., laptop, mobile phone, tablet device), smart device (e.g., smartphone, smart watch, activity tracker, smart apparel, smart accessory), security and/or monitoring device, a server, a router, a network computer, a peer device, edge device or other common network node, and so on. In some optional embodiments, a smart phone, tablet, or computer (i.e., a laptop or desktop computer) can comprise both the computing device 200 and the display 1011. Alternatively, it is contemplated that the display 1011 can be provided as a separate component from the computing device 200.

The remote computing device 200 may comprise one or more processors 1003, a system memory 1012, and a bus 1013 that couples various components of the computing device 200 including the one or more processors 1003 to the system memory 1012. In the case of multiple processors 1003, the computing device 200 may utilize parallel computing.

The bus 1013 may comprise one or more of several possible types of bus structures, such as a memory bus, memory controller, a peripheral bus, an accelerated graphics port, and a processor or local bus using any of a variety of bus architectures.

The computing device 200 may operate on and/or comprise a variety of computer readable media (e.g., non-transitory). Computer readable media may be any available media that is accessible by the computing device 200 and comprises, non-transitory, volatile and/or non-volatile media, removable and non-removable media. The system memory 1012 has computer readable media in the form of volatile memory, such as random access memory (RAM), and/or non-volatile memory, such as read only memory (ROM). The system memory 1012 may store data such as fertility data 1007 and/or program modules such as operating system 1005 and device/fertility software 1006 that are accessible to and/or are operated on by the one or more processors 1003.

The computing device 200 may also comprise other removable/non-removable, volatile/non-volatile computer storage media. A mass storage device 1004 may provide non-volatile storage of computer code, computer readable instructions, data structures, program modules, and other data for the computing device 200. The mass storage device 1004 may be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like.

Any number of program modules may be stored on the mass storage device 1004. An operating system 1005 and the device/fertility software 1006 may be stored on the mass storage device 1004. One or more of the operating system 1005 and the device/fertility software 1006 (or some combination thereof) may comprise program modules and the device/fertility software 1006. Fertility data 1007 may also be stored on the mass storage device 1004. The fertility data 1007 may be stored in any of one or more databases known in the art. The databases may be centralized or distributed across multiple locations within the network 1015.

A user may enter commands and information into the computing device 200 via an input device (not shown). Such input devices comprise, but are not limited to, a keyboard, pointing device (e.g., a computer mouse, remote control), a microphone, a joystick, a scanner, tactile input devices such as gloves, and other body coverings, motion sensor, and the like These and other input devices may be connected to the one or more processors 1003 via a human machine interface 1002 that is coupled to the bus 1013, but may be connected by other interface and bus structures, such as a parallel port, game port, an IEEE 1394 Port (also known as a Firewire port), a serial port, network adapter 1008, and/or a universal serial bus (USB).

A display 1011 may also be connected to the bus 1013 via an interface, such as a display adapter 1009. It is contemplated that the computing device 200 may have more than one display adapter 1009 and the computing device 200 may have more than one display 1011. A display 1011 may be a monitor, an LCD (Liquid Crystal Display), light emitting diode (LED) display, television, smart lens, smart glass, and/or a projector. In addition to the display 1011, other output peripheral devices may comprise components such as speakers (not shown) and a printer (not shown) which may be connected to the computing device 200 via Input/Output Interface 1010. Any step and/or result of the methods may be output (or caused to be output) in any form to an output device. Such output may be any form of visual representation, including, but not limited to, textual, graphical, animation, audio, tactile, and the like. The display 1011 and computing device 200 may be part of one device, or separate devices.

The computing device 200 may operate in a networked environment using logical connections to one or more remote computing devices 1014a,b,c (i.e., computing devices that are remote from computing device 200). A remote computing device 1014a,b,c may be a personal computer, computing station (e.g., workstation), portable computer (e.g., laptop, mobile phone, tablet device), smart device (e.g., smartphone, smart watch, activity tracker, smart apparel, smart accessory), security and/or monitoring device, a server, a router, a network computer, a peer device, edge device or other common network node, and so on. Logical connections between the computing device 200 and a remote computing device 1014a,b,c may be made via a network 1015, such as a local area network (LAN) and/or a general wide area network (WAN). Such network connections may be through a network adapter 1008. A network adapter 1008 may be implemented in both wired and wireless environments. Such networking environments are conventional and commonplace in dwellings, offices, enterprise-wide computer networks, intranets, and the Internet. In further exemplary aspects, it is contemplated that the computing device 200 can be in communication with the remote computing devices 1014a,b,c through a Cloud-based network. In exemplary aspects, it is contemplated that data from computing device 200 can be transmitted wirelessly to other remote computing devices 1014a,b,c for use by clinicians or other individuals involved with the health care of the device user. Optionally, in these aspects, the computing device 200 can allow a user to selectively upload fertility data to a Cloud storage unit, from which the remote computing devices 1014a,b,c can securely access a patient's fertility information. In further aspects, it is contemplated that the fertility data can be provided as a report or dataset that can be downloaded by a clinician for use in providing further advice or treatment to the patient.

Application programs and other executable program components such as the operating system 1005 are shown herein as discrete blocks, although it is recognized that such programs and components may reside at various times in different storage components of the computing device 200, and are executed by the one or more processors 1003 of the computing device 200. An implementation of the device/fertility software 1006 may be stored on or sent across some form of computer readable media. Any of the disclosed methods may be performed by processor-executable instructions embodied on computer readable media.

Exemplary Aspects

In view of the described products, systems, and methods and variations thereof, herein below are described certain more particularly described aspects of the invention. These particularly recited aspects should not however be interpreted to have any limiting effect on any different claims containing different or more general teachings described herein, or that the “particular” aspects are somehow limited in some way other than the inherent meanings of the language literally used therein.

Aspect 1: A menstrual cycle monitoring device comprising: an intravaginal ring including at least one sensing probe and/or sensor oriented to measure bioelectrical impedance, resistance or conductance; a data communication unit that is configured to store and transmit collected fertility data which includes at least the measured bioelectrical impedance, resistance, and/or conductance over time; a temperature sensor configured to measure body temperature and communicate the measured body temperature to the data communication unit; and a power source electrically coupled to the sensing probe(s) and/or sensor(s) and the data communication unit.

Aspect 2: The device of aspect 1, wherein the intravaginal ring further comprises an annular body sized to be oriented in the vaginal vault and/or the vaginal environment proximal to the cervix.

Aspect 3: The device of aspect 2, wherein the annular body is formed of medical-grade silicone or polyurethane.

Aspect 4: The device of aspect 2 or aspect 3, wherein the power source is selectively removable from the annular body.

Aspect 5: The device of any one of the preceding aspects, wherein the sensing probe(s) and/or sensor(s) includes a complementary set of electrodes.

Aspect 6: The device of aspect 5, wherein the complementary set of electrodes have a gold electrode surface.

Aspect 7: The device of any one of the preceding aspects, wherein the sensing probe(s) and/or sensor(s) further includes an antimicrobial coating.

Aspect 8: The device of any one of the preceding aspects, wherein the data communication unit further comprises a wireless transmitter configured to send the fertility data to a remote computing device for access by the user.

Aspect 9: A system comprising: the menstrual cycle monitoring device of any one of the preceding aspects; and a remote computing device having a processor that is configured to receive collected fertility data from the data communication unit of the menstrual cycle monitoring device.

Aspect 10: The system of aspect 9, wherein the remote computing device is selected from the group consisting of a smart phone, a tablet, a smart watch, and a computer.

Aspect 11: The system of aspect 9 or aspect 10, wherein the processor of the remote computing device is configured to execute a tracking application that analyzes and displays fertility estimates based on the fertility data.

Aspect 12: The system of aspect 11, wherein the tracking application of the remote computing device is configured to determine hydration based upon the fertility data.

Aspect 13: The system of aspect 12, wherein the tracking application is configured to correlate the measured bioelectrical impedance, resistance, and/or conductance of the collected fertility data with a corresponding hydration of cervical mucus.

Aspect 14: The system of aspect 12 or aspect 13, wherein the sensing probe(s) and/or sensor(s) of the device comprises a sensor configured to measure bioelectrical resistance.

Aspect 15: The system of aspect 12 or aspect 13, wherein the sensing probe(s) and/or sensor(s) of the device comprises a sensor configured to measure bioelectrical conductance.

Aspect 16: The system of aspect 12 or aspect 13, wherein the probe(s) and/or sensor(s) of the device comprises a sensor configured to measure bioelectrical impedance.

Aspect 17: A method comprising: using the system of claim 9; orienting the intravaginal ring such that the at least one hydration and/or ion sensing probe(s) and/or sensor(s) is in fluid communication with cervical fluid of a subject; and using the data communication unit to transmit fertility data of the subject to the remote computing device.

Aspect 18: The method of aspect 17, wherein the remote computing device is selected from the group consisting of a smart phone, a tablet, a smart watch, and a computer

Aspect 19: The method of aspect 17 or aspect 18, further comprising, using the processor of the remote computing device to analyze and display fertility estimates based on the fertility data.

Aspect 20: The method of claim 19, wherein the processor of the remote computing device is configured to determine hydration based upon the fertility data.

The foregoing detailed description describes the invention with reference to specific exemplary embodiments. However, it can be appreciated that various modifications and changes can be made without departing from the scope of the present invention as set forth in the appended claims. The detailed description and accompanying drawings are to be regarded as merely illustrative, rather than as restrictive, and all such modifications or changes, if any, are intended to fall within the scope of the present invention as described and set forth herein.

1. A menstrual cycle monitoring device comprising;
a) an intravaginal ring including at least one sensing probe and/or sensor oriented to measure bioelectrical impedance, resistance or conductance;
b) a data communication unit that is configured to store and transmit collected fertility data which includes at least the measured bioelectrical impedance, resistance, and/or conductance over time;
c) a temperature sensor configured to measure body temperature and communicate the measured body temperature to the data communication unit; and
d) a power source electrically coupled to the sensing probe(s) and/or sensor(s) and the data communication unit.

2. The device of claim 1, wherein the intravaginal ring further comprises an annular body sized to be oriented in the vaginal vault and/or the vaginal environment proximal to the cervix.

3. The device of claim 2, wherein the annular body is formed of medical-grade silicone or polyurethane.

4. The device of claim 2, wherein the power source is selectively removable from the annular body.

5. The device of claim 1, wherein the sensing probe(s) and/or sensor(s) includes a complementary set of electrodes.

6. (canceled)

7. The device of claim 1, wherein the sensing probe(s) and/or sensor(s) further includes an antimicrobial coating.

8. The device of claim 1, wherein the data communication unit further comprises a wireless transmitter configured to send the fertility data to a remote computing device for access by the user.

9. A system comprising:
a) a menstrual cycle monitoring device having:
i. an intravaginal ring including at least one sensing probe and/or sensor oriented to measure bioelectrical impedance, resistance or conductance;
ii. a data communication unit that is configured to store and transmit collected fertility data which includes at least the measured bioelectrical impedance, resistance, and/or conductance over time;
iii. a temperature sensor configured to measure body temperature and communicate the measured body temperature to the data communication unit; and
iv. a power source electrically coupled to the sensing probe(s) and/or sensor(s) and the data communication unit; and
b) a remote computing device having a processor that is configured to receive fertility data that is collected by the data communication unit of the menstrual cycle monitoring device.

10. The system of claim 9, wherein the remote computing device is selected from the group consisting of a smart phone, a tablet, a smart watch, and a computer.

11. The system of claim 9, wherein the processor of the remote computing device is configured to execute a tracking application that analyzes and displays fertility estimates based on the fertility data.

12. The system of claim 11, wherein the tracking application of the remote computing device is configured to determine hydration based upon the fertility data.

13. The system of claim 12, wherein the tracking application is configured to correlate the measured bioelectrical impedance, resistance, and/or conductance of the collected fertility data with a corresponding hydration of cervical mucus.

14. The system of claim 12, wherein the sensing probe(s) and/or sensor(s) of the device comprises a sensor configured to measure bioelectrical resistance.

15. The system of claim 12, wherein the sensing probe(s) and/or sensor(s) of the device comprises a sensor configured to measure bioelectrical conductance.

16. The system of claim 12, wherein the probe(s) and/or sensor(s) of the device comprises a sensor configured to measure bioelectrical impedance.

17. A method comprising:
a) using a menstrual cycle monitoring device having:
i. an intravaginal ring including at least one sensing probe and/or sensor oriented to measure bioelectrical impedance, resistance or conductance;
ii. a data communication unit that is configured to store and transmit collected fertility data which includes at least the measured bioelectrical impedance, resistance, and/or conductance over time;
iii. a temperature sensor configured to measure body temperature and communicate the measured body temperature to the data communication unit; and
iv. a power source electrically coupled to the sensing probe(s) and/or sensor(s) and the data communication unit;
b) orienting the intravaginal ring such that the at least one hydration and/or ion sensing probe(s) and/or sensor(s) is in fluid communication with cervical fluid of a subject; and
c) using the data communication unit of the device to transmit fertility data of the subject to a device positioned external of the subject.

18. The method of claim 21, wherein the remote computing device is selected from the group consisting of a smart phone, a tablet, a smart watch, and a computer

19. The method of claim 21, further comprising using a processor of the remote computing device to analyze and display fertility estimates based on the fertility data.

20. The method of claim 19, wherein the processor of the remote computing device is configured to determine hydration based upon the fertility data.

21. The method of claim 17, further comprising using a remote computing device to receive the fertility data.
